(19) United States
(12) Patent Application Publication
(10) Pub. No.: US 2005/0014147 A1
(43) Pub. Date: Jan. 20, 2005

(54) Method and apparatus for three label microarrays

(76) Inventor: Martin J Hessner, Brookfield, WI (US)

Correspondence Address: QUARLES & BRADY LLP, 411 E. WISCONSIN AVENUE, SUITE 2040, MILWAUKEE, WI, 53202-4497, US

(21) Appl. No.: 10/486,244

(22) PCT Filed: Aug. 16, 2002
(86) PCT No.: PCT/US02/26302

Related U.S. Application Data
(60) Provisional application No. 60/314,005, filed on Aug. 21, 2001.

(51) Int. Cl. C12Q 1/68; G01N 33/53

(52) U.S. Cl. 435/6; 435/7.1

(57) ABSTRACT

A method of and apparatus for directly visualizing printed microarrays are disclosed. In one embodiment, the method comprises the steps of (a) generating labeled probes labeled with a first label, (b) constructing a microarray with the labeled probes, wherein the microarray comprises a plurality of probe spots, and (c) examining the microarray to determine the amount of probe present at each probe spot.

### CROSS-REFERENCE TO RELATED APPLICATION

The application claims priority to U.S. Ser. No. 60/314,005, filed Aug. 21, 2001 and incorporated by reference.

### STATEMENT REGARDING FEDERALLY SPONSORED RESEARCH OR DEVELOPMENT

### BACKGROUND OF THE INVENTION

The cDNA microarray platform has great potential to generate new insights into human disease (Dhanasekaran, et al., 2001; Garber, et al., 2001; Hedenfalk, et al., 2001; Hegde, et al., 2001; Schena, et al., 1995; Schena, et al., 1996; Sorlie, et al., 2001). The use of cDNA microarrays begins with construction of the array, where typically, hundreds to thousands of cDNA probes are amplified by PCR, purified, and printed onto coated glass slides (typically poly-L-lysine or amino saline). In a typical experiment, slides are fixed, blocked, and are finally hybridized with Cy3- and Cy5-labeled cDNA targets derived from the two biological samples being compared for differential gene expression. After hybridization, the array is analyzed with a fluorescence scanner and the relative amounts of an mRNA species in the original two samples is defined as a ratio between the two fluorophores at the homologous array element using specially designed software (Eisen and Brown, 1999; Hegde, et al., 2000; Schena, et al., 1995; Schena, et al., 1996; Wang, et al., 2001).

This useful technology, however, possesses recognized data quality/reproducibility issues, that can limit its application to complex biological systems (Kerr and Churchill, 2001; Lee, et al., 2000). High experimental variability can arise through laboratory technical problems as well as normal biological variation (Pritchard, et al., 2001). Yue, et al., (2001), using Saccharomyces cerevisiae probes and complementary in vitro transcripts, demonstrated that the amount of DNA bound to the glass slide is dependent, in part, on the concentration of the DNA printed and that the amount retained by the slide is critical for good quality differential expression data (Yue, et al., 2001). The range of detected values of known transcript ratios was compressed when elements were printed at concentrations less than 100 ng/ul in water. Printing at more dilute printing concentrations exacerbated ratio compression to the point where input transcript ratios of 30:1 or 1:30 were detected as output ratios close to 1:1, illustrating that limiting bound probe results in an underestimation or failure to detect differential gene expression (Yue, et al., 2001). The concentration of DNA printed, the printing buffer selected, and the glass coating will influence the amount of DNA retained by the slide after processing. Commonly used printing solutions include 3×SSC (saline sodium citrate), 50% dimethyl sulfoxide (DMSO), and water (Eisen and Brown, 1999; Yue, et al., 2001). Diehl, et al., (2001) found that the addition of the PCR additive betaine, which is known to normalize base pair stability differences, increase solution viscosity, and reduce evaporation rates, also greatly enhances probe binding to poly-L-lysine coated slides (Diehl, et al., 2001; Henke, et al., 1997; Rees, et al., 1993). Furthermore, probe saturation of the glass slide was obtained at a lower printing concentration of 250 ng/ul when betaine was present versus >500 ng/ul in printing solutions without betaine, which can greatly increase the number of potential slides produced from a single library amplification (Diehl, et al., 2001).

### DESCRIPTION OF THE DRAWINGS

FIG. 1 is an evaluation of spotting solutions for post-blocking probe retention. Dilutions series fluorescein-labeled cDNA probe was printed in five different printing FIG. 1A: Fluorescein image immediately after printing. FIG. 1B: Fluorescein image (same array as panel A) after aqueous post-processing. FIG. 1C: Plotted are the percent retention values determined from the 100 ng/μl dilution element for each of the 3 genes for the 6 printing solutions (n=35 elements, distributed over 35 slides). Bar graphs ordered: GAPDH; B-actin, HBGR2. 3% DMSO 1.5M betaine was superior.

FIG. 2 indicates that the processed array fluorescein image is reflective of hybridized array performance. The experiment employs human 10K probe cDNA arrays. FIG. 2A1-A3 (nonaqueous blocking)/A4-6 (aqueous blocking): Array image immediately after printing (A1, A4), post processing (A2, A5), and homotypic hybridization with Cy5 and Cy3 direct labeled UACC903 RNA. FIG. 2B: Scatter plots of homotypic hybridizations on arrays processed with nonaqueous (top) and aqueous (bottom) methods. FIG. 2C: The variability in intensity Cy3/Cy5 ratio measurement (y-axis) is correlated with fluorescein signal to noise ratio (x-axis); nonaqueous (top) and aqueous (bottom) methods. Images were collected using same laser and PMT settings and are illustrated under the same parameters using GenePix Pro Software. [Note: Loss of DNA after processing step (A1 vs A2; A4 vs A5); white elements in panels A1 and A4 are saturated]

FIG. 3 demonstrates that fluorescein signal to noise score (x-axis) of 50 replicate pairs (100 slides) is predictive of correlation coefficient of Cy3/Cy5 ratio data between hybridized replicate arrays (y-axis). All hybridizations are between Jurkat and UACC903 cDNA.

FIG. 4 demonstrates a tracking scheme for confirmation of plate order and orientation from clone source plate to printed array using fluorescein labeled probes. Panel A: Layout of asymmetric plate-specific negative controls for first 4 clone source plates. Position Al of each plate is removed to serve as an orientation marker; a second negative control is used as a plate identifier. Panel B: 9600 element human cDNA array printed on in-house-prepared poly-L-lysine coated slide using 16 pins (set back). Subarrays generated by each pin are labeled. Subarray 1 possesses position A1 from each source plate (A1 negative controls generate the absence of 24/25 elements in the first (far left) column. Subarray 9 (enlarged) shows a correct series of negative controls for indicated plates; other probe plates are represented in other subarrays. Improper management of any plate at any point during array construction will disrupt this pattern. Note: observable pin clogging problem on pin 2.

FIG. 5 shows a linear relationship between amount of labeled DNA deposited on slide (x axis) and fluorescence detected (y axis). To accomplish this, multiple (n=4) serial dilutions in water (400 ng/ul to 0.049 ng/ul) were generated from a pooled DNA sample derived from 384 separate cDNA clone amplifications to account for different clone sizes (for example, single clones, one of 500 bp and one of 2000 bp, each at a concentration of 150 ng/ul will have a molarity difference of 4-fold, and therefore a difference in fluorescence of 4-fold). Known volumes possessing known quantities (0.5 ul) of DNA were hand spotted on to poly-L-lysine slides, dried, and imaged. Fluorescein relative fluorescence units (RFU) were plotted against picograms of DNA (FIG. 2) to determine that, with the Packard ScanArray 5000 (laser power 70%; PMT 80%), there are approximately 25 RFU detected per picogram DNA in this experiment. Average spot size in this experiment was >1500 microns in diameter with total DNA deposited being 50, 100, 200, 400, and 800 pg for the points represented. Based upon a printing concentration of 150 ng/ul, a probe deposition volume of 0.6 nl, and an 80% retention rate with our new buffer, we estimate that approximately ˜75 pg of DNA is retained and available for hybridization. From these array elements, which measure ˜120 microns in diameter, we typically detect 10,000 RFU or 133 RFU per picogram, a discrepancy of approximately 5-fold. We know that fluorescein when in close proximity will self-quench, perhaps this is why the detected fluorescence on mechanically generated spot is less than we would expect based on this experiment.

FIG. 6A demonstrates the use of fluorescein-labeled cDNA probes to evaluate spot/array morphology after printing and after fixing and blocking for in-house prepared versus commercially note differences spot morphology and probe retention. Arrays 1-4 were printed on poly-L-lysine coated slides produced at the Medical College of Wisconsin; Electron Microscopy Sciences, Fort Washington, Pa.; Polysciences Inc., Warrington, Pa.; Cel-Associates, Pearland, Tex., respectively. Arrays 5-13 were printed on aminosaline coated slides produced by Asper Biotech, Redwood City, Calif.; Apogent Discoveries, Waltham, Mass.; Bioslide Technologies, Walnut, Calif.; Erie Scientific, Portsmouth, N.H.; Genetix, St. James, N.Y.; Coming Inc, Corning N.Y. (GAPS); Corning Inc, Corning N.Y. (GAPS II); Sigma, St. Louis, Mo.; Telechem International Inc, Sunnyvale, Calif., respectively. Arrays 14-15 were printed on epoxy coated slides produced by Telechem International Inc, Sunnyvale, Calif. (epoxy and super epoxy, respectively). FIG. 6B demonstrates competitive hybridization between Jurkat (Cy5) and UACC903 (Cy3) labeled cDNA (30 ug total RNA labeled though incorporation of Cy5 or Cy3-dUTP) hybridized to 10K human arrays printed on 16 different coated slides. Arrays 1-4 were printed on poly-L-lysine coated slides produced by MCW; Electron Microscopy Sciences, Fort Washington, Pa.; Polysciences Inc., Warrington, Pa.; Cel-Associates, Pearland, Tex., respectively. Arrays 5-13 were printed on aminosaline coated slides produced by Asper Biotech, Redwood City, Calif.; Apogent Discoveries, Waltham, Mass.; Bioslide Technologies, Walnut, Calif.; Erie Scientific, Portsmouth, N.H.; Genetix, St. James, N.Y.; Corning Inc, Corning N.Y. (GAPS); Corning Inc, Corning N.Y. (GAPS II); Sigma, St. Louis, Mo.; Telechem International Inc, Sunnyvale, Calif., respectively. Arrays 14-15 were printed on epoxy coated slides produced by Telechem International Inc, Sunnyvale, Calif. (epoxy and super epoxy, respectively).

FIG. 7 demonstrates imaging of a fluorescein-labeled oligonucleotide (70-mer) after printing (1A, 2A, 3A) and after fixing/blocking (2A, 2B, 2C) in three different spotting solutions (A: 1.5M betaine/3% DMSO; B: 3×SSC; C: 50% DMSO). Addition of a third color is useful for quality control of cDNA arrays as well as spotted oligonucleotide arrays.

### SUMMARY OF THE INVENTION

In one embodiment, the invention is a method of directly visualizing printed microarrays, comprising the steps of: (a) generating labeled probes labeled with a first label, (b) constructing a microarray with the labeled probes, wherein the microarray comprises a plurality of probe spots, and (c) examining the microarray to determine the amount of probe present at each probe spot. In one preferred form of the invention, the labeled probes are either cDNA or oligonucleotides and the first label is fluorescent. In another embodiment, the labeled probes are proteins or antibodies.

In one embodiment, the labeled probes are labeled with a fluorescent probe, such as fluorescein, and the examination of step (c) is via the detection of relative fluorescence units and is by the use of a confocal laser scanner.

In one embodiment of the invention, the labeled DNA probes are between 10 and 100,000 base pairs in length and the probes comprise 1 fluorescent label molecule per DNA strand on average.

In another embodiment, the invention comprises the method described above additionally comprising the steps of (d) exposing the microarray to labeled target molecules, wherein the labeled target molecules are labeled with a second and third label, preferably a fluorescent label, and (e) examining the microarray to determine the amount of target hybridized to the probes.

In another embodiment, the invention is a microarray comprising (a) a surface and (b) labeled DNA probes attached to the surface in a plurality of spots, wherein each probe is labeled with a first fluorescent label.

### DESCRIPTION OF THE INVENTION

Controlling array fabrication variables is difficult because the array printing is typically invisible until after hybridization. In the present invention, we have generated labeled probe arrays, as a means of visualizing element/array morphology and quantifying DNA deposition/retention on the slide prior to hybridization. Direct labeling of probes separates slide coating, printing, and processing from hybridization and facilitates evaluation and optimization of methods. We have made the observation that slides coated, printed and processed together are not necessarily equivalents, and that prehybridization imaging is predictive of hybridization performance. Therefore, prehybridization slide evaluation and selection can improve data reproducibility and quality because slides that do not meet minimum standards can be avoided.

A number of approaches have been described to address the problem of determining DNA deposition/retention and array element morphology prior to experimental use of slides. It is possible to stain the fixed slide prior to hybridization with a DNA-binding fluorescent dye, such as SYBR Green II or SYTO 61 (Battaglia, et al., 2000; Yue, et al., 2001). However, investigational use of the slide after quality control analysis requires destaining, and potential changes in slide performance after destaining must be considered. The use of universal targets which will hybridize to every element of a microarray have also been reported (Yue, et al., 2001). While these hybridization-based techniques provide information as to the amount of DNA present within each element of the array, they require sacrificing a slide from a batch of printed slides for quality control analysis and do not completely assure the investigator that the arrays actually used for experimentation are equivalent to those evaluated during quality control. Most recently, Ramakrishnan, et al., 2002 describe co-spotting a fluorescent dye with as a component of the printing buffer for monitoring mechanical aspects of array fabrication. In this approach, the label is not covalently attached to the probe, and the spiked dye is presumably washed off during blocking and fixing steps, so one does not know much probe was retained on the array since the array is again invisible.

To circumvent this problem, we developed a means of directly visualizing printed arrays by generating probes labeled with a fist label, preferably labeled with fluorescent-label primers such as fluorescein-labeled primers (excitation 488 nm/emission 508 nm), which are spectrally compatible with the Cy5 and Cy3 dyes typically used for target labeling (Cy3 excitation 543 nm/emission 570 nm; Cy5 excitation 633 nm/emission 670 nm) when using the GSI Luminonics ScanArray 5000 confocal laser scanner. The narrow 10 nm bandwidth of this instrument allows for excitation of Cy3 at 543 nm without co-excitation of fluorescein, which would contaminate the Cy3 emission with its broad emission tail. One might also wish to use luminescent or phosphorescent dyes. One may wish to use radioactive dyes. It is necessary that the first label be covalently coupled to the probe and that the first label be detectable and spectrally compatible. These probes are deposited, preferably as described below, in spots on a microarray surface, preferably a coated glass slide. By “spots”, we mean a deposit (or “printing”) of probes in discrete, specific area, such that hybridization of labeled targets to that specific area can be detected.

By “spectrally compatible” we mean that the trio of dyes are detectable and distinct from each other in a confocal laser scanner. Fluorescein, Cy5 and Cy3 are spectrally compatible using the GSI Luminonics ScanArray 5000 confocal laser scanner. Other trios of dyes would be equally suitable with this and other scanning systems. Other trios would include any combination of fluorescein derivatives for lowest wavelength dye, including Alexafluor 488 (Molecular Probes, Eugene Oreg.). The Alexafluor homologues for Cy3 and Cy5 could also be used for the middle and high wavelength dyes.

Our approach, which separates analysis of slide coating, printing, and processing from analysis of hybridization provides a method for 1) probe amplification control, 2) direct examination of array/element morphology, 3) determination of post-processed probe retention, and 4) a means of bound probe quantitative quality control for improved differential gene expression analysis.

An advantage of this approach is the existence of a direct relationship between detected relative fluorescence units (RFUs) and the amount of DNA probe present on the slide, once unincorporated primer has been removed from the amplified probe, making DNA retention studies possible.

The present invention is a method and apparatus for performing a microarray analysis. In one embodiment, the method comprises creating a cDNA microarray wherein the cDNA is labeled with a first label, preferably a fluorescent label. Preferably, this first label is fluorescein. The first label must be spectrally compatible with second and third labels. Target molecules are labeled with either the second or third labels.

Microarrays can be fabricated using either amplified cDNAs as a source of probe material or, alternatively, a synthetic oligonucleotide. Oligonucleotide arrays, currently fall into two categories, those that are fabricated through in situ synthesis, where the oligonucleotide probe is synthesized directly on the array surface (example Affymetrix GeneChip, which uses 25-mers); or a spotted oligonucleotide array, where the fully synthsized oligo is spotted onto the array surface and attached through a variety of different chemstries (these oligos are typically longer, i.e., 70-mers). The spotted oligo arrays offer the advantage of being able to purify the probe that actually is attached to the array (i.e., removal of short molecules that failed during synthesis), currently offer more flexibility in design, and can be fabricated in the research laboratory. We envision that the present invention would encompass “spotted oligonucleotide” arrays. When we refer the microarrays comprising “oligonucleotides,” we are referring to creation of full-length oligonucleotides that are then spotted onto the array.

Since synthetic oligonucleotides are made in a 3′ to 5′ direction, the addition of a compatible dye to the 5′-most position will result in the labeling of only full-length molecules. A label of this nature would be useful to spotted arrays since one could determine how much full-length oligonucleotide was present at each position on the array, as well as assess other array parameters, such as spot shape. In the case of spotted oligq arrays, it would be possible to measure how probe was redistributed over the array during the blocking steps, as we have described for cDNA arrays.

It is possible to label proteins with dyes (including radioactive ones) for this same purposes. Therefore, the present invention comprises protein and antibody arrays. One would be able to confirm that the protein is present, how much, shape of spot, and how well the protein contained within the spot.

In one embodiment of the invention, one would examine the labeled microarray and directly measure the bound probe via detection of the first label. The Examples below describe preferable methods for this analysis. All the probes must be labeled. The cDNAs are typically generated by PCR from plasmid clones. Labeling of this PCR product is accomplished through the use of oligonucleotide primers that are 5′ end-labeled with the first label. Since the primer becomes part of the PCR product, the cDNA is essentially covalently labeled once on each 5′ end. Such primers for use in PCR sequencing, etc., are readily available from oligonucleotide vendors. After analysis, one would be able to discard microarrays that are that are not consistent a preset quality control standard. One might identify, in general, how much bound is necessary to obtain highly reproducible results across high density arrays. However, for key experiments, we are selecting arrays with signal to noise ratios >0.90, average element fluorescein intensity >3,000, and CV (coefficient of variation) of element fluorescein intensity <10%.

In another embodiment of the present invention, one would expose the microarray described above to the labeled targets and perform a microarray binding analysis.

In another embodiment of the present invention, a microarray is provided wherein the probe is labeled with a first label. Preferably this label is fluorescent and the array is either a cDNA or an oligonucleotide array. In another embodiment of the present invention, the array is a protein array or an antibody array.

The array of the present invention is preferably created by the following steps: The cDNA array is typically prepared by first amplifying by PCR the cDNA clone inserts from their plasmid vectors. This can be done in a 96-well format or a 384-well format. We use 384-format for PCR and all subsequent steps. Clones that serve as a source of cDNA templates can be commercial vendor, such as Research Genetics or the I.M.A.G.E. Consortium, or personal cDNA libraries. PCR reactions to amplify these cDNA clone inserts can be conducted directly from bacterial culture or from purified plasmid template. In either case, the oligonucleotide primers are labeled with a first fluorescent label. We have selected fluorescein due to our instrumentation and its compatibility with Cy3 and Cy5 on our instrumentation. After PCR of the 20,000-plus clones to be printed on the chip, the PCR reactions must be purified. This is done for a number of reasons, including removing PCR reaction components and buffer. We have chosen a size exclusion filtration approach since it removes most of the unincorporated labeled oligonucleotide. After purification, the 384 plate is quantified, dried down, and reconstituted in 1.5M betaine/3%DMSO for printing. Probe material is then printed onto coated glass slides as “spots”. Since the PCR product has been purified, and unincorporated labeled primers are removed, the measured fluorescence on the array is proportional to the amount of PCR product present on the slide versus due to PCR product plus primer. This approach is different than other visualization methods because the probe is covalently attached to the label, versus a staining interaction or hybridization. This method allows every slide to have QC analysis before use.

The preferred first label is fluorescein or a fluorescein derivative. Fluorescein derivatives have been the most commonly used label for biological molecules. In addition to its relatively high absorption properties, excellent fluorescence quantum yield and good water solubility, fluorescein has an excitation maximum (494 nm) that closely matches the 488 nm spectral line of the argon-ion laser, making it a useful fluorophore for confocal laser-scanning microscopy applications. Our selection of fluorescein as the “first label” was first driven by fact that it is compatible with Cy3 and C5 when using the ScanArray 5000, and second by the fact that this fluorophore is relatively inexpensive and readily available as a 5′ end-label on oligonucleotide primers.

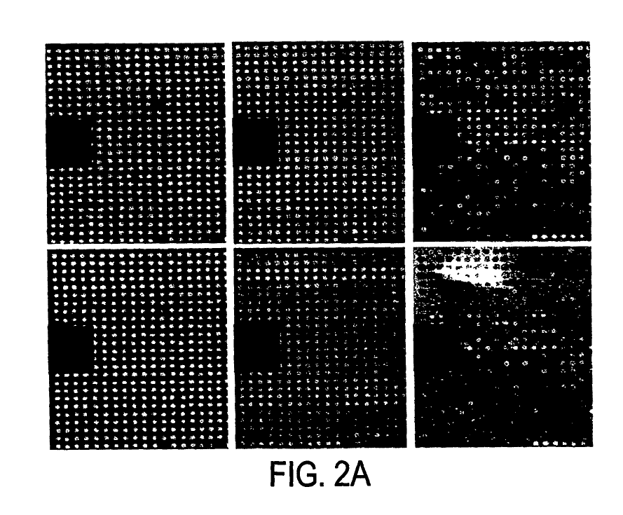

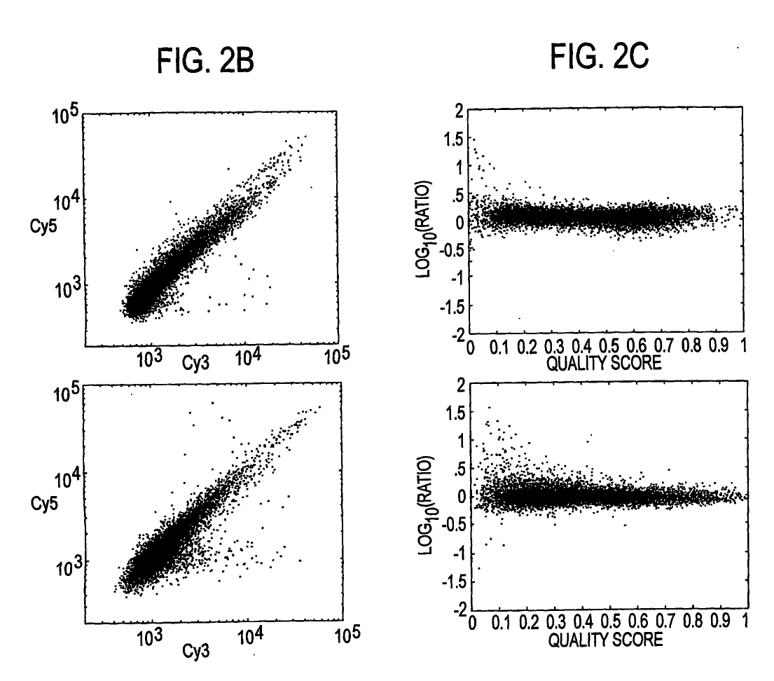

Unfortunately, many confocal laser scanners do not possess the performance specifications to support the use of a three-color system as we describe here using fluorescein. In our system, the following excitation/emission wavelengths are used: Fluorescein 488 nm/508 nm; Cy3 543 nm/570 nm; Cy5 633 nm/670 nm. The key feature of the Scan Array 5000 instrument that makes 3 dyes possible, besides the fact that it has the required laser to excite fluorescein at 488 nm, is the fact that it can excite and read these wavelengths with a very narrow bandwidth (±5 nm). Practically, this means that Cy3 can be excited without co-exciting fluorescein; since fluorescein has such a broad emission spectrum, if it were to be excited when trying to excite Cy3, the Cy3 emission spectrum would be contaminated. This situation is likely to change as both the fluorescent labels and instrumentation continue to improve, allowing more flexibility in dye and instrument selection in three-color applications. None the less, the strategy as described in this report performs well. We are confident fluorescent labeling of the probes does not interfere with the subsequent detection of second and third label (Cy3 and C5) hybrids, because (1) scanning of slides prior to hybridization shows no signal for either the second or third label (Cy3 or Cy5 in our Example); and (2) second/third label (Cy3/Cy5) scatter plots pass through the origin with no evidence of the detected second or third label (Cy3 or Cy5) signal being negatively influenced by a quenching effect nor positively influenced by carryover signal. Furthermore, all of our arrays (including those shown in FIGS. 2, 4, 6A and 6B) possess a series of fluorescein-labeled Arabidopsis thaliana probes to be used as positive (in combination with homologous in vitro transcript) and negative controls. These probes generate no signal under second or third label (Cy3 or Cy5 in our Example) scanning conditions either before or after hybridization in the absence of labeled in vitro transcript.

Direct measurement of the bound probe available for hybridization has other important advantages. Electrophoretic analysis of probe amplification efficiency can be greatly reduced since failed PCRs can be identified and recorded through analysis of fluorescein signal intensity. Precious clinical target material can be conserved through reduction of replicates necessary because poor quality slides can be avoided. Quality-based prehybridization selection results in a higher probability of successful experiments and reduced overall cost. Preferably, we select arrays with signal to noise ratios >0.90, average element fluorescein intensity >3,000, and CV (coefficient of variation) of element fluorescein intensity <10%.

In one version of the present invention, one would introduce targets labeled with second and third labels. In a preferred embodiment, the method would comprise the following steps: RNA samples are isolated from the tissues that are being compared for gene expression. Labeled cDNA targets are derived from these samples by reverse transcription, whereby Cy 3 is incorporated into one sample and Cy5 is incorporated into the other. Equal amounts of the two labeled samples are hybridized to the array, allowing the labeled targets to base pair with their respective homologous probe on the array. The array is the washed and scanned for both wavelengths in a confocal laser scanner and the images analyzed by software. Transcripts in both samples in equal amounts will give rise to dye ratios of “1”; whereas transcripts over or under expressed relative to the other sample will give rise to ratios deviating from one.

### EXAMPLES

### Example 1

### Three Color cDNA Microarrays: Quantitative Assessment through the use of Fluorescein Labeled Probes

Results:

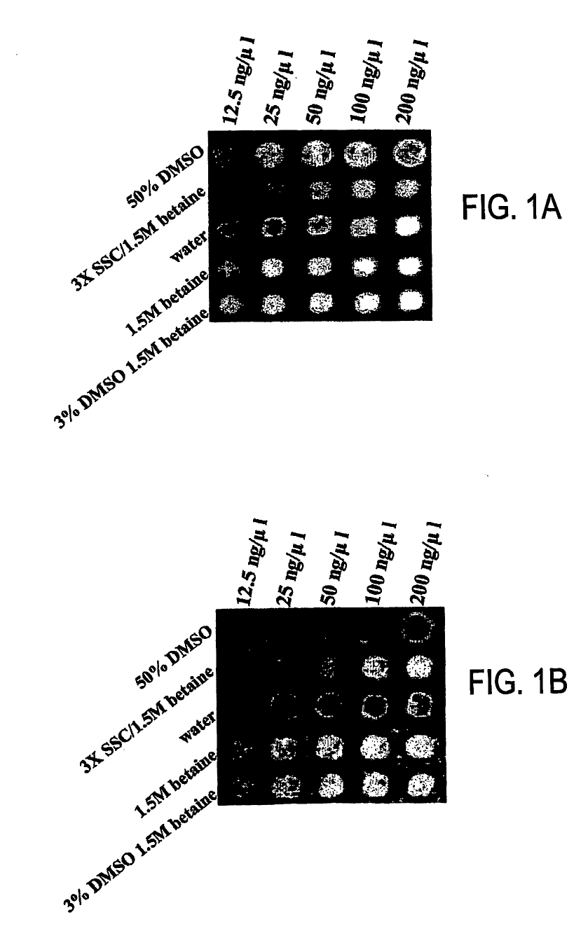

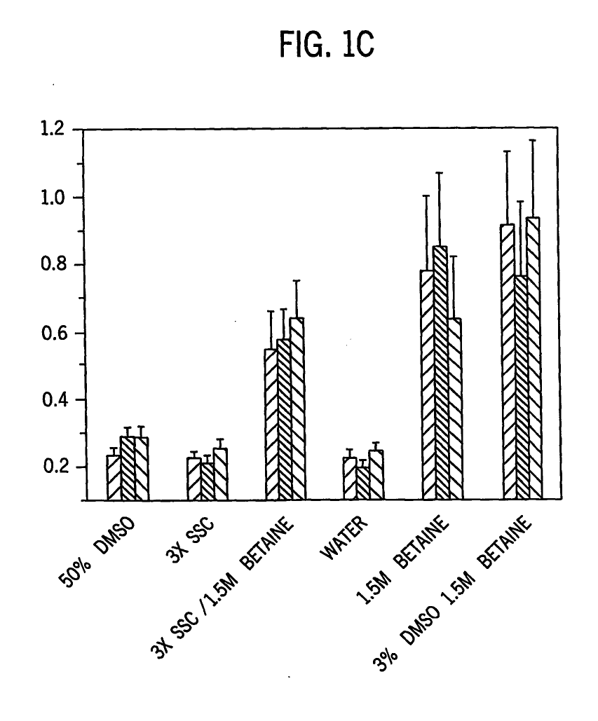

Human probes for glyceraldehyde 3-phosphate deydrogenase-1 (GAPDH), B-actin, and glutamate receptor-2 (HBGR2) (IMAGE Consortium 50117, 34357, and 43622, respectively) were serially diluted and printed in 50% DMSO, 3×SSC, water, 1.5M betaine, 1.5M betaine/3×SSC (Diehl, et al., 2001) and 1.5M betaine/3.1% DMSO. Arrays were evaluated for spot morphology (size/shape) and DNA retention was measured by scanning arrays immediately after printing and again after post-processing. Only 30% of probe is retained by poly-L-lysine coated glass slides after post-processing when the commonly used printing solutions water, 50% DMSO, or 3×SSC are used [FIGS. 1A and 1B]. Probes printed with 50% DMSO resulted in 151.1±5.9 micron diameter array elements compared to 120.6±5.4 micron diameter elements for those printed in water or 3×SSC (with or without 1.5M betaine), therefore, DMSO was titrated in an effort to control spot size. The use of 3% DMSO/1.5M betaine resulted in the highest average probe retention on the slide (>70%), more than twice what is observed with commonly used printing solutions, as well as optimal average spot size (<130 microns) [FIG. 1C]. Preparation of DNA probe is the most time consuming and expensive component of high-density array construction and making efficient use of prepared probe through high retention an important ongoing issue.

The critical post-arraying blocking process, where unreacted primary amines are converted to carboxylic moieties, is typically performed with succinic anhydride in an aqueous borate buffered 1-methyl-2-pyrrolidinone (Dolan, et al., 2001; Eisen and Brown, 1999; Schena, et al., 1995; Schena, et al., 1996). Generation of fluorescein-labeled arrays enabled direct hybridization-free comparison of this traditional blocking process to blocking with succinic anhydride in the non-polar, non-aqueous solvent 1,2-dichloroethane (Diehl, et al., 2001). Processing with the nonaqueous method resulted in arrays with very low background fluorescein signal levels compared to the aqueous blocking method [FIG. 2A2 versus 2A5] where background levels increased as a function of printed DNA concentration (data not shown). The prehybridization image quality was predictive of slide performance in homotypic hybridizations employing UACC903 RNA where arrays processed with the nonaqueous method generated images with higher overall signal intensity and fewer outliers [2A3 versus 2A6, 2B].

Image quality was assessed with Matarray software (Wang, et al., 2001), which employs a spatial and intensity dependent algorithm for spot detection and signal segmentation. Matarray also generates a composite quality score (q_{com}) that is defined for each spot on the array according to size, signal-to-noise value (signal/signal+noise), background uniformity and saturation status (Wang, et al., 2001). Variation in Cy5/Cy3 intensity ratio values correlated with the fluorescein q_{com} score and revealed an overall lower spot quality with the nonaqueous method that impacts data quality [FIG. 2C]. Using simultaneously produced 10,000 probe arrays, mean signal to noise quality score (signal/[signal+noise]) per element of 0.93±0.04 (n=15) were observed with the non-aqueous method versus 0.71±0.02 (n=15) with the aqueous method. Probe signal measurements of 6-9 fold over noise were observed on arrays processed with the nonaqueous blocking method and values slightly less for those arrays aqueously processed; these values are sufficient for credible measurement of bound probe. These observations are consistent with the notion that aqueous blocking methods result in partial re-dissolving and re-deposition of printed DNA, generating higher background.

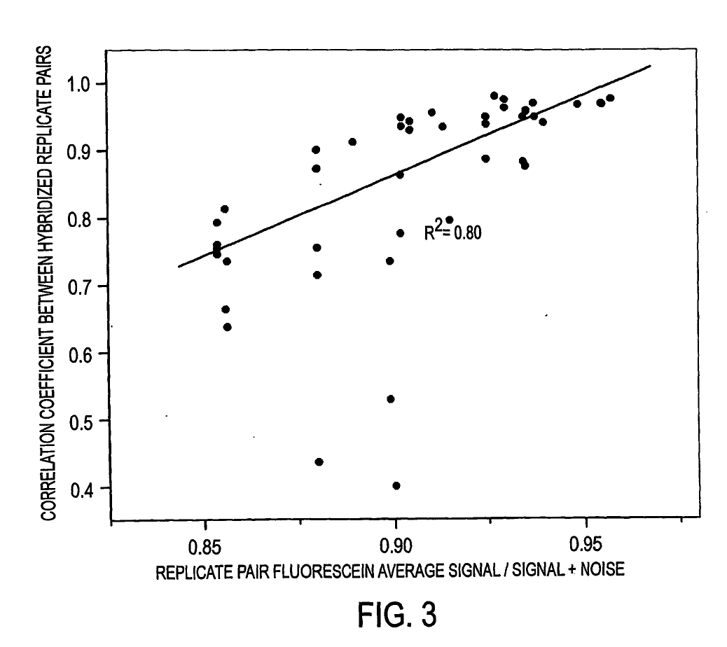

Slides that are coated, printed, and processed together do not necessarily result in equivalent arrays. One hundred slides each possessing a 10,000 human probe array were simultaneously printed, nonaqueously processed, and evaluated. The average fluorescein signal/slide varied between processed slides from 4,500 RFU to 20,000 RFU (10,770±4,202); while overall slide signal to noise values ranged from 0.85 to 0.95 (mean=0.92±0.03). Competitive hybridizations between UACC903 and Jurkat cDNA on arrays, selected from three independent printings of the same probe set, with high DNA/element and low background values were compared to those performed on arrays with low DNA/element and/or high background values. When comparing hybridization results between replicate pairs of differing quality (n=50 pairs), a direct and significant relationship (R²=0.80, p<0.001) was observed between prehybridization fluorescein image quality and replicate consistency, illustrating that microarray data quality can be improved through prehybridization slide selection based upon quality analysis. The observation of a relationship between pre and post hybridized image/data quality is completely consistent with our previous report in that prehybridized arrays possessing low signal to noise scores give rise to hybridized arrays with low signal to noise scores and hybridization data from such arrays do not correlate well with each other (Wang, et al., 2001). Selection of quality arrays does not necessarily guarantee high replicate Cy5/Cy3 ratio correlation, because RNA samples, target labeling, hybridization, washing, laboratory technique, and image collection are sources of variation, as indicated by the three outliers observed in FIG. 3. It must be emphasized that the 100 hybridizations represented in FIG. 3 were performed by multiple laboratory personnel utilizing multiple labeling reactions of the same RNA.

Methods:

The Research Genetics (Huntsville, Ala.) sequence-verified human library, consisting of 41,472 clones was used as a source of probe DNA. The library was reformatted from 96 to 384-format and subsequently manipulated using 0.5 μl and 5 μl volume 96 and 384 slot pin replicator tools (VP Scientific, San Diego, Calif.). Clone inserts were directly amplified in 384-well format from 0.5 μl bacterial culture using 0.26 μM of each vector primer [array F: 5′-fluorescein-CTGCMGGCGAT-(fluorescein)TAAGTTGGGTMC-3′ (SEQ ID NO:1) and array R: 5′-fluorescein-GTGAGCGGAT-(fluorescein)MCAAMTTTCACACAGGAACAGC-3′ (SEQ ID NO:2)] (Integrated DNA Technologies, Coralville, Iowa) in a 20 μl reaction consisting of 10 mM Tris-HCl pH8.3, 3.0 mM MgCl₂, 50 mM KCl, 0.2 mM each dNTP (Amersham, Piscataway, N.J.), 1M betaine, and 0.25 U Taq polymerase (Roche, Indianapolis Ind.). Reactions were incubated at 95° C. for 5 minutes and 35 cycles of 95° C. for 1 minute, 55° C. for 1 minute, 72° C. for 1 minute, and terminated with a 7 minutes hold at 72°. PCR products were routinely analyzed for quality by 1% agarose gel electrophoresis analysis. Products were purified by size exclusion filtration using the Multiscreen 384 PCR filter plates (Millipore, Bedford, Mass.) to remove unincorporated primer and PCR reaction components. Forty wells of each 384-well probe plate were quantified by the PicoGreen assay (Molecular Probes, Eugene, Oreg.) according to the manufacturers instructions, dried down, and reconstituted at 125 ng/μl in 3% DMSO/1.5M betaine.

Microarrays possessing a density of 10,000 probes/slide were printed onto poly-L-lysine slides using a GeneMachines Omni Grid printer (San Carlos, Calif.) with 8 Telechem International SMP3 pins (Sunnyvale, Calif.). Slides were post-processed using the previously described aqueous (Eisen and Brown, 1999) or nonaqueous (Diehl, et al., 2001) protocols. Slide coating, isolation of mRNA, labeling, and hybridization were performed as described previously in Hedge, et al., 2000; Schena, et al., 1995; and Yue, et al., 2001. After hybridization, arrays were scanned with a ScanArray 5000 (GSI Luminonics, Billerica, Mass.) and image files were obtained. Array image files were analyzed with the Matarray software (Wang, et al., 2001).

### Example 2

### Use of a Three-Color cDNA Array Platform to Measure and Control Available Bound Probe for Improved Data Quality and Reproducibility

We directly evaluated the impact of differing amounts of bound probe on hybridized replicate data correlation, and investigated the performance of 15 different vendor-supplied coated slides in terms of DNA retention and hybridization performance. Furthermore, utilizing our three-color cDNA microarray platform, we developed and describe here a novel probe tracking system for ascertainment of proper plate order and orientation from culture growth, amplification, and purification, through printing of probes onto the array.

Materials and Methods:

Library Growth and Tracking

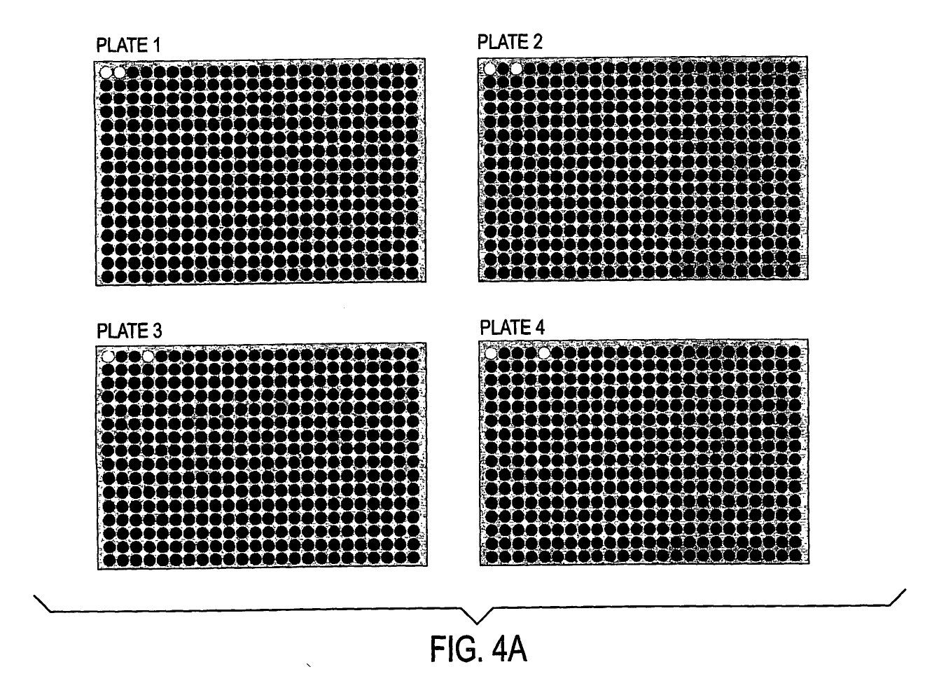

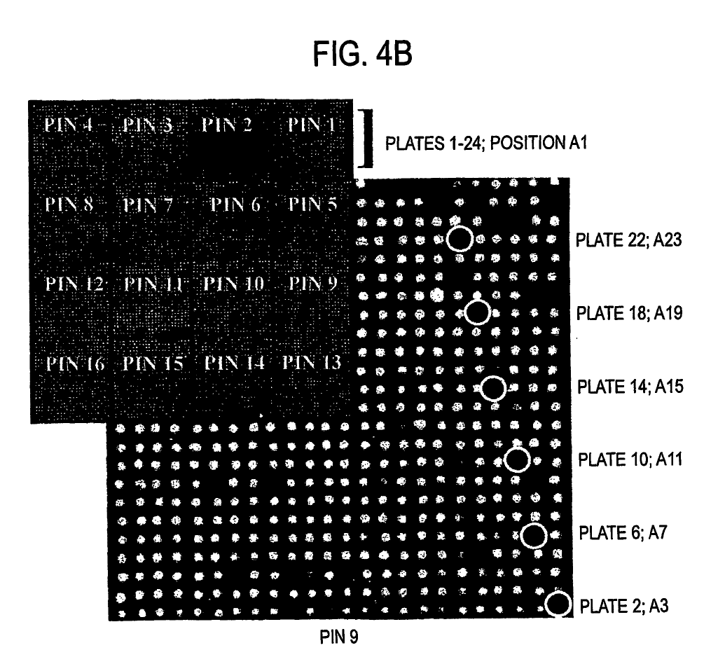

The Research Genetics (Huntsville, Ala.) sequence-verified human library, consisting of 41,472 clones was used as a source of probe DNA. The library was reformatted from 96 to 384-format and subsequently manipulated using 0.5 μl and 5 μl volume 96 and 384 slot pin replicator tools (VP Scientific, San Diego, Calif.). Cultures were grown in 150 ul Terrific Broth (Sigma, St. Louis, Mo.) supplemented with 100 mg/ml ampicillin in 384 deep-well plates (Matrix Technologies, Hudson, N.H.) sealed with air pore tape sheets (Qiagen, Valencia, Calif.) and incubated with shaking for 16-18 hours. A unique asymmetric pattern of two negative controls per 384 culture plate was created by transferring the contents of the selected wells to a new 384 plate and updating the clone tracking database accordingly. The plate-specific negative control pattern was created by removing position A1 (to establish an orientation marker) and one additional plate-specific wellA2 (FIG. 4).

Clone inserts were amplified in duplicate in 384-well format from 0.5 ul bacterial culture diluted 1:8 in sterile distilled water or from 0.5 ul purified plasmid (controls only) using 0.26 μM of each vector primer {SK865 5′-fluorescein-GTC CGT ATG TTG TGT GGA A-3′ (SEQ ID NO:3) and SK536: 5′-fluorescein-GCG AAA GGG GGA TGT GCT G-3′ (SEQ ID NO:4) (Yue, et al., 2001)} (Integrated DNA Technologies, Coralville, Iowa) in a 20 μl reaction consisting of 10 mM Tris-HCl pH 8.3, 3.0 mM MgCl₂, 50 mM KCl, 0.2 mM each dNTP (Amersham, Piscataway, N.J.), 1M betaine (Henke, et al., 1997; Rees, et al., 1993) and 0.50 U Taq polymerase (Roche, Indianapolis Ind.). Reactions were amplified with a touchdown thermal profile consisting of 94° C. for 5 minutes; 20 cycles of 94° C. for 1 minute, 60° C. for 1 minute (minus 0.5° per cycle), 72° C. for 1 minute; and 15 cycles of 94° C. for 5 minutes; 20 cycles 94° C. for 1 minute, 55° C. for 1 minute, 72° C. for 1 minute; terminated with a 7 minutes hold at 72° (Don, et al., 1991; Hecker and Roux, 1996; Roux and Hecker, 1997). PCR products were routinely analyzed for quality by 1% agarose gel electrophoresis analysis. Products from replicate plates pooled and then purified by size exclusion filtration using the Multiscreen 384 PCR filter plates (Millipore, Bedford, Mass.) to remove unincorporated primer and PCR reaction components. Forty wells of each 384-well probe plate were quantified by the PicoGreen assay (Molecular Probes, Eugene, Oreg.) according to the manufacturers instructions; alternatively, 1 ul of each 384 plate well was pooled and absorbance at 260 nm read directly for quantification. After quantification, all plates were dried down, and reconstituted at 125 ng/μl in 3% DMSO/1.5M betaine.

Poly-L-lysine coated slides were prepared in-house as previously described (Eisen and Brown, 1999). Nine different commercially available aminosaline coated slides (Apogent Discoveries, Waltham, Mass.; Asper Biotech, Redwood City, Calif.; Bioslide Technologies, Walnut, Calif.; Corning Inc, Corning N.Y.; Erie Scientific, Portsmouth, N.H.; Genetix, St. James, N.Y.; Sigma, St. Louis, Mo.; Telechem International Inc, Sunnyvale, Calif.) and 3 different commercially available poly-L-lysine coated slides (Cel-Associates, Pearland, Tex.; Electron Microscopy Sciences, Fort Washington, Pa.; Polysciences Inc., Warrington, Pa.) were obtained for evaluation. Lastly, two types of epoxy-coated slide (Telechem International Inc, Sunnyvale, Calif.), and slides coated with a proprietary chemistry obtained from Full Moon Biosystems (Sunnyvale, Calif.) were obtained. In all 16 different slide sources, including poly-L-lysine slides prepared in-house, belonging to 3 general categories, were evaluated in terms of spot morphology and DNA retention.

Microarrays possessing a density of 9,600 human probes/slide were printed onto coated slides using a GeneMachines Omni Grid printer (San Carlos, Calif.) with 16 Telechem International SMP3 pins (Sunnyvale, Calif.) at 40% humidity and 22° C. (72° F.). To control pin contact force and duration, the instrument was set with the following Z motion parameters, velocity: 7 cm/sec, acceleration: 100 cm/sec², deceleration: 100 cm/sec².

Slides were post-processed using the previously described nonaqueous protocol (Diehl, et al., 2001). Slide coating, isolation of mRNA, labeling, and hybridization were performed as described previously in Hedge, et al., 2000; Schena, et al., 1995; and Yue, et al., 2001. Image files on all arrays were collected after printing (fluorescein), after blocking (fluorescein), and again after hybridization (Cy3 and Cy5) with a ScanArray 5000 (GSI Luminonics, Billerica, Mass.). Array image files were analyzed with the Matarray software (Wang, et al., 2001).

Results and Discussion. Quality array construction requires generation of adequate amounts concentrated probe and printing probes in a known ordered fashion onto coated glass slides. We have opted to reformat libraries from 96 to 384-format for culture growth/archiving, PCR, purification, and printing. This has reduced the number of plates of our 41,472 human clone library from 432 to a more manageable 108. A highly optimized touchdown PCR protocol has been developed whereby 1-2 ug purified probe material is recovered from 2 pooled and purified 20 ul PCR reactions. Duplicate reactions compensate for random PCR failures, enabling overall PCR success rates, based upon gel analysis, of ˜90%. Recovery of >1 ug purified probe enables printing >2000 arrays per amplification (assuming: 4 ul plate dead volume, printing at 150 ng/ul, and 250 nl/pickup/100 slides using the TeleChem SMP3 pins). The fact that the array is visible prior to hybridization allows for spots that are not present on the array due to PCR failure or mechanical problems (clogged pin) to be tracked, eliminating a potential source of error/variance between replicate slides. This has lead to the development of a tracking system, which utilizes a unique pattern of negative controls for each clone source plate enabling a means to assess that all plates have had order and orientation maintained from the clone source plate through growth, PCR, pooling, purification, and finally printing (FIG. 4).

A number of critical parameters, including DNA concentration, printing buffer, slide surface, temperature, humidity, and print head velocity can influence the amount of DNA deposited, retained, and ultimately available for hybridization on the slides surface (Diehl, et al., 2001; Yue, et al., 2001; Hegde, et al., 2000). Previously, we evaluated the retention characteristics of 50% DMSO, 3×SSC, water, 1.5M betaine, 1.5M betaine/3×SSC and 1.5M betaine/3.1% DMSO on poly-L-lysine coated slides prepared in our own laboratory and found that on this surface, 1.5M betaine/3% DMSO offered the best retention (˜70%) under the conditions described in the Methods section. Since printing of labeled probes enables direct measurement of DNA deposition and retention, we evaluated 15 different commercially available coated slides, in an attempt to identify surfaces that offered the best performance in terms of background fluorescence, spot morphology, amount of DNA ultimately available for hybridization, and competitive hybridization performance using Cy3 and Cy5 labeled Jurkat and UACC903 cDNA. Including our in-house prepared slides, 18 different prepared surfaces were available for comparison: poly-L-lysine (n=4), aminosaline (n=9), epoxy (n=2), and a single unknown proprietory chemistry (Full Moon Biosystems; Sunnyvale, Calif.). A single 9600 element human cDNA array was spotted onto each slide in 1.5M betaine/3% DMSO; additionally, a 384 plate of human cDNA probes in water, 3×SSC, and 50% DMSO were spotted onto each slide in order to control for the possibility that some of the commercial surfaces may have been optimized for spotting with these more commonly used solutions. Five replicate arrays for each slide type were generated. These five replicates were evenly distributed over the arrayer deck (capacity 100 slides) by arranging the slides into 5 groups of 18 to account for any variance introduced by placement in the print order (ie first versus last). Prior to printing, background Cy3, Cy5, and fluorescein fluorescence was measured. Fluorescein background was observed on all poly-L-lysine slides except for those produced in-house. Fluorescein background was also observed on 6 of aminosaline slides (Asper Biotech, Corning, Erie Scientific, Genetix, Telechem), as well as on the proprietary surface from Full Moon Biosciences. Cy3 background was again observed on all 3 commercial poly-L-lysine slides but not those prepared in-house. No Cy3 background was observed on any of the aminosaline or epoxy slides. Slight Cy5 background was observed on only 2 commercial poly-L-lysine slides (Electron Microscopy Sciences, Polysciences Inc.).

Fluorescein images were obtained immediately after printing and again after post-processing to measure DNA deposited and retained. This required a confocal laser scanner calibration method; to ensure consistent image collection, therefore we set the laser voltage power on the instrument (typically ˜70%) against the FluorIS (CLONDIAG, Jena, Germany), a non-bleaching, reusable, calibration/standardization tool for fluorescein, Cy5, and Cy3 image collection, while holding the photo multiplier tube (PMT) parameters constant (80%). Under these conditions, multiple scans of the same array are possible with little to no detectable fluorescein signal degradation.

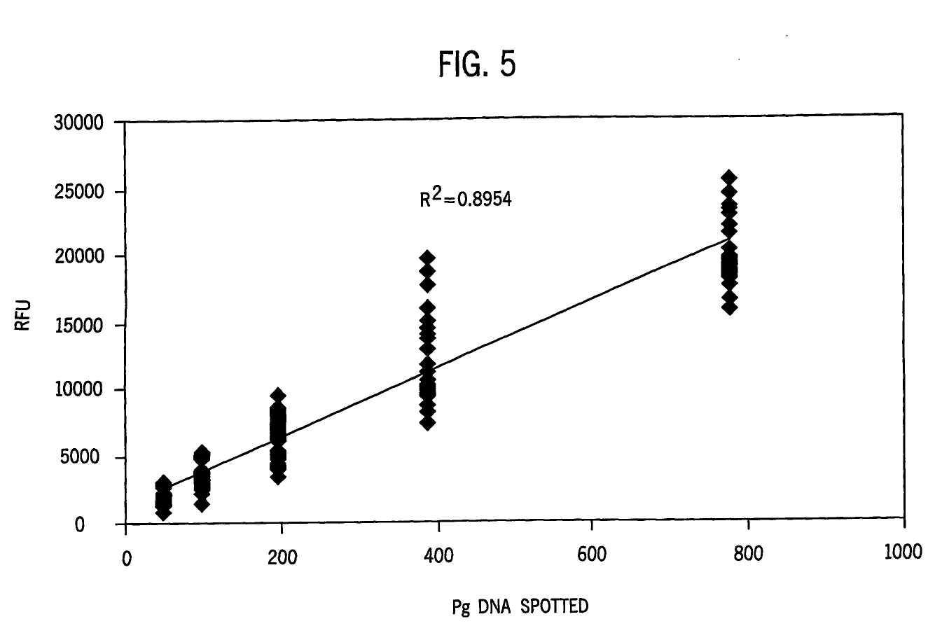

PCR products amplified from cDNA clones using single-labeled oligonucleotide primers possess two dyes per double-stranded product and product sizes typically range from ˜500 bp to ˜2000 bp. Therefore, it is possible to mathematically predict the amount of fluorescence generated per picogram of amplified and purified PCR product. However, a direct measurement avoids the error introduced through variables such as fluorescein-fluorescein proximity quenching effects. To accomplish this, multiple (n=4) serial dilutions in water (x ng/ul to y ng/ul) were generated from a pooled DNA sample derived from 384 separate cDNA clone amplifications to account for different clone sizes. Known volumes possessing known quantities of DNA were spotted on to poly-L-lysine slides, dried, and imaged. Fluorescein relative fluorescence units (RFU) were plotted against picograms of DNA (FIG. 5) to determine that, with the Packard ScanArray 5000 (laser power 70%; PMT 80%), there are approximately Z picograms/RFU.

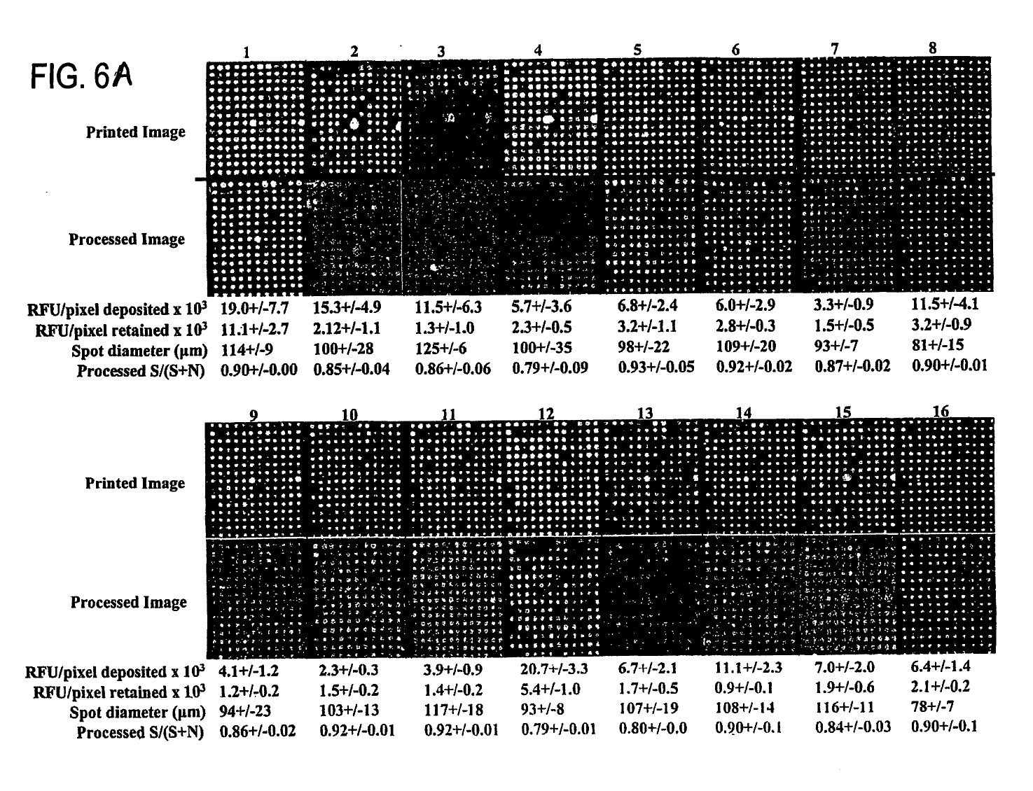

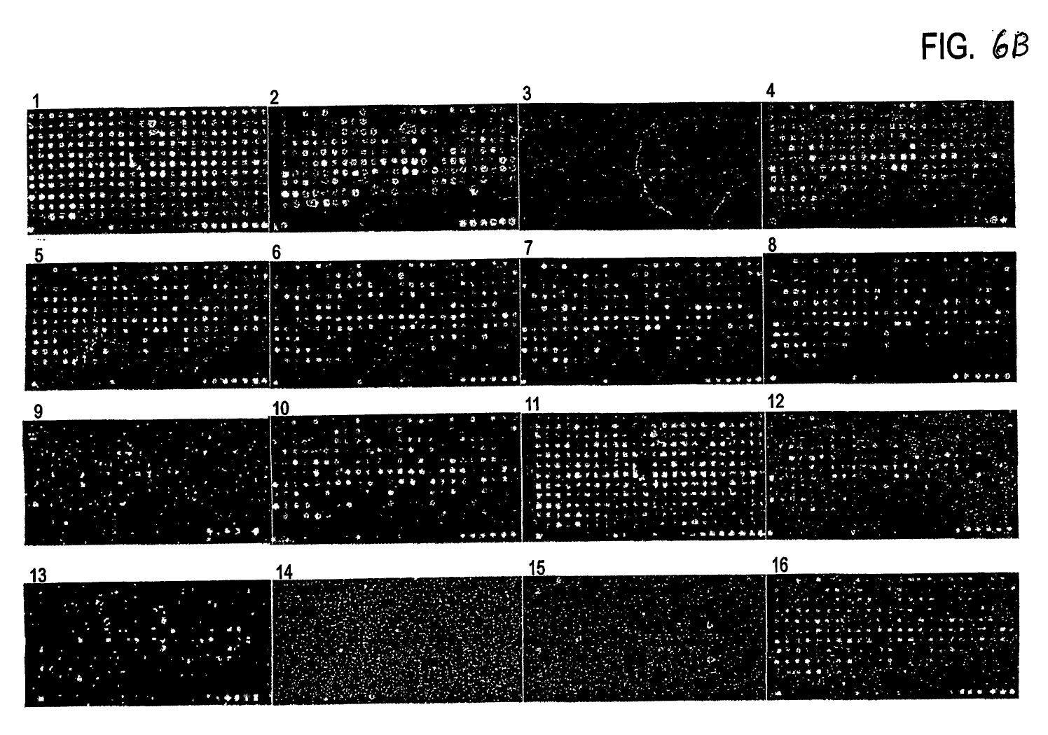

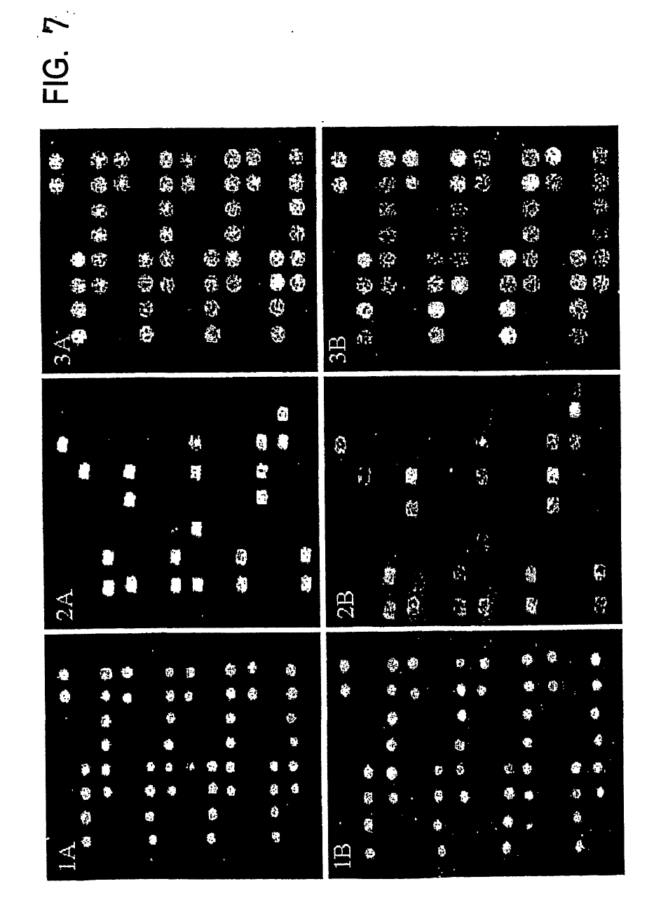

Illustrated in FIG. 6 are images of human cDNA arrays possessing 9600 elements spotted on the 16 different coated surfaces using 10% DMSO/1.5M betaine as a printing buffer. Images of arrays immediately after printing (FIG. 6A), after processing (FIG. 6B), and after competitive hybridization to labeled Jurkat and UACC903 cDNA (FIG. 6C) are shown. All hybridizations were prepared from a single pool of labeled cDNAs to normalize any variances introduced through individual reverse transcription reactions. This experiment illustrates that not all vendor supplied coated slides are, equivalent and probe labeling can be used to measure the amount of material available on the array surface.

To further evaluate the impact of the amount of bound probe available on the overall quality of gene expression data obtained from cDNA microarrays, two hundred 9600 element human cDNA probes were printed onto 100 slides with a single pin loading per probe. This resulted in a series of arrays with an average bound probe per element available for hybridization ranging from X pg/element to Y pg/element. The overall goal of this experiment was to establish a general guideline as to how much DNA is needed per element to ensure that probe is in excess relative to labeled target for the majority of transcripts one may encounter in a standard microarray experiment. This would enable the future identification of those arrays possessing insufficient bound probe, which as replicates would introduce experimental variability. This series of arrays was hybridized again to a pool of labeled Jurkat and UACC903 cDNAs to normalize any differences between individual target labeling reactions.

References:

Battaglia, C., G. Salani, C. Consolandi, L. R. Bernardi and G. De Bellis, 2000 Analysis of DNA microarrays by non-destructive fluorescent staining using SYBR green 11. Biotechniques 29:78-81.
Dhanasekaran, S. M., T. R. Barrette, D. Ghosh, R. Shah, S. Varambally et al., 2001 Delineation of prognostic biomarkers in prostate cancer. Nature 412:822-826.
Diehl, F., S. Grahlmann, M. Beier and J. Hoheisel, 2001 Manufacturing DNA microarrays of high spot homogeneity and reduced background signal. Nucleic Acids Research 29:e38.
Dolan, P. L., Y. Wu, L. K. Ista, R. L. Metzenberg, M. A. Nelson et al., 2001 Robust and efficient synthetic method for forming DNA microarrays. Nucleic Acids Res 29:E107-107.
Don, R. H., et al., ‘Touchdown’ PCR to circumvent spurious priming during gene amplffication. Nucleic Acids Res, 1991.19(14):4008.
Eisen, M., and P. Brown, 1999 DNA arrays for analysis of gene expression. Methods in Enzymology 303:179-205.
Garber, M. E., O. G. Troyanskaya, K. Schluens, S. Petersen, Z. Thaesler et al., 2001 Diversity of gene expression in adenocarcinoma of the lung. Proc Natl Acad Sci USA 98:13784-13789.
Hecker, K. H. and K. H. Roux, High and low annealing temperatures increase both specificity and yield in touchdown and stepdown PCR. Biotechniques, 1996. 20(3):478-85.
Hedenfalk, I., D. Duggan, Y. Chen, M. Radmacher, M. Bittner et al., 2001 Gene-expression profiles in hereditary breast cancer. N Engl J Med 344:539-548.
Hegde, P., R. Qi, K. Abernathy, C. Gay, S. Dharap et al., 2000 A concise guide to cDNA microarray analysis. Biotechniques 29:548-550, 552-544, 556 passim.
Hegde, P., R. Qi, R. Gaspard, K. Abernathy, S. Dharap et al., 2001 Identification of tumor markers in models of human colorectal cancer using a 19,200-element complementary DNA microarray. Cancer Res 61:7792-7797.
Henke, W., K. Herdel, K. Jung, D. Schnorr and S. Loening, 1997 Betaine improves the PCR amplification of GC-rich sequences. Nucleic Acids Research 25:3957-3958.
Hessner, M. J., K. Dowling, O. Kokanovic, L. Meyer, S. H. Nye, X. Wang, J. Waukau, S. Ghosh, Use of fluorescein-labeled probes as a quality control tool for cDNA microarrays. The American Journal of Human Genetics, 2001. Supplement, 69:468.
Hessner, M. J., et al., Three color cDNA microanrays: Quantitative assessment through the use of fluorescein-labeled probes. Submitted, 2002.
Kerr, M., and G. Churchill, 2001 Bootstrapping cluster analysis: Assessing the reliability of conclusions from microarray experiments. Proc Natl Acad Sci 98:8961-8965.
Lee, M., F. Kuo, G. Whitmore and J. Sklar, 2000 Importance of replication in microarray gene expression studies: Statistical methods and evidence from repetitives cDNA hybridizations. Proc Natl Acad Sci 97:9834-9839.
Pritchard, C. C., L. Hsu, J. Deirow and P. S. Nelson, 2001 Project normal: defining normal variance in mouse gene expression. Proc Natl Acad Sci USA 98:13266-13271.
Ramakrishnan, et al., Nucleic Acid Res 30:e30, 2002.
Rees, W., T. Yager, J. Korte and P. Von Hippel, 1993 Betaine can eliminate the base pair composition dependence of DNA melting. Biochemistry 32:137-144.
Roux, K. H. and K. H. Hecker, One-step optimization using touchdown and stepdown PCR. Methods Mol Biol, 1997. 67:39-45.
Schena, M., D. Shalon, R. W. Davis and P. O. Brown, 1995 Quantitative monitoring of gene expression patterns with a complementary DNA microarray. Science 270:467-470.
Schena, M., D. Shalon, R. Heller, A. Chai, P. O. Brown et al., 1996 Parallel human genome analysis: microarray-based expression monitoring of 1000 genes. Proc Natl Acad Sci USA 93:10614-10619.
Sorlie, T., et al., 2001 Gene expression patterns of breast carcinomas distinguish tumor subclass with clinical implications. Proc Natl Acad Sci 98:10869-10874.
Wang, X., S. Ghosh and S.-W. Guo, 2001 Quantitative quality control in microarray image processing and data acquisition. Nucleic Acids Research 29:E75-82.
Yue, H., P. S. Eastman, B. B. Wang, J. Minor, M. H. Doctolero et al., 2001 An evaluation of the performance of cDNA microarrays for detecting changes in global mRNA expression. Nucleic Acids Res 29:E41-41.

1. A method of directly visualizing microarrays, comprising the steps of:
a) generating labeled probes labeled with a first label,
b) constructing a microarray with the labeled probes, wherein the microarray comprises a plurality of probe spots, and
c) examining the microarray to determine the amount of probe present at each probe spot.

2. The method of claim 1 wherein the probes are DNA molecules.

3. The method of claim 1 wherein the probes are selected from the group consisting of cDNA and oligonucleotides.

4. The method of claim 1 wherein the probe is selected from the group consisting of proteins and antibodies.

5. The method of claim 1 wherein the labeled probes are attached to the microarray surface via electrostatic and covalent bonds.

6. The method of claim 1 wherein the first label is fluorescent.

7. The method of claim 1 wherein the labeled probes are labeled with fluorescein.

8. The method of claim 1 wherein the label is selected from the group consisting of fluorescent, radioactive, phosphorescent and luminescent labels.

9. The method of claim 5 wherein the examination of step (c) is via the detection of relative fluorescence units and is by the use of a confocal laser scanner.

10. The method of claim 1 wherein a preferred amount of probe has been determined and the microarrays are evaluated using this preset amount.

11. The method of claim 5 wherein the fluorescently labeled probes of step (a) are generated via labeled primers.

12. The method of claim 2 wherein the labeled probes are between 10 and 100,000 base pairs in length.

13. The method of claim 2 wherein the probes comprise 1 label molecules per DNA strand on average.

14. The method of claim 1 additionally comprising the step of
(d) exposing the microarray to labeled target molecules wherein the labeled target molecules are labeled with a second and third label.

15. The method of claim 14 comprising the additional step of
(e) examining the microarray to determine the amount of target bound to the probes.

16. The method of claim 1 wherein the microarray comprises a poly-lysine-coated glass slide.

17. The method of claim 2 wherein DMSO/1.5 M betaine is used during the attachment of the probes to the microarray.

18. The method of claim 1 wherein step (c) comprises measurement of image quality as assessed by software which employs a spatial and intensity-dependent algorithm for spot detection and signal segmentation.

19. The method of claim 1 wherein the microarrays possess a density of 3,000-10,000 probes/slide.

20. A printed microarray comprising
a) a surface, and
b) labeled probes attached to the surface in a plurality of spots, wherein each probe is labeled with a first label, wherein the probe is selected from the group consisting of spotted oligonucleotides, cDNA, protein and antibodies.

21. The microarray of claim 20 wherein the probe is DNA.

22. The microarray of claim 20 wherein the probe is selected from the group consisting of nucleic acids, protein, and antibodies.

23. The array of claim 20 wherein the surface is a glass slide.

24. The array of claim 20 wherein the surface is coated with a coating selected from the group consisting of poly-L-lysine, aminosaline, epoxy, and aminoallyl.

25. The array of claim 20 wherein the first label is fluorescent.

26. The array of claim 25 wherein the first fluorescent label is fluorescein.

27. The array of claim 20 wherein the first label is selected from the group consisting of fluorescent, luminescent, radioactive or phosphorescent labels.
